(19) United States
(12) Patent Application Publication
(10) Pub. No.: US 2026/0250757 A1
(43) Pub. Date: Aug. 27, 2026

(54) SYSTEMS AND METHODS FOR DETECTING PESTICIDE RESISTANCE

(71) Applicant: CLARKE MOSQUITO CONTROL PRODUCTS, INC., St. Charles, IL (US)

(72) Inventors: Mariana Lizbeth Jiménez Martinez, Monterey (MX); Iram Pablo Rodríguez Sánchez, Monterey (MX); Gerardo de Jesús Trujillo Rodríguez, Monterey (MX); Everarado González González, Monterey (MX)

(21) Appl. No.: 19/065,668

(22) Filed: Feb. 27, 2025

(51) Int. Cl. C12Q 1/6865; G01N 21/29

(52) U.S. Cl. CPC C12Q 1/6865; G01N 21/29; C12Q 2600/124; C12Q 2600/178

(57) ABSTRACT

Methods and detection kits for identification of pesticide resistance in pests. The kits may be suitable for the detection of resistance in the field as they allow a quick and simple identification of a resistance marker with a small sample size. Further disclosed are methods for resistance management in pests that are resistant to certain pesticides.

### FIELD

This disclosure relates to systems and methods for detecting resistance of pests to a pesticide. Exemplary methods and systems may be used in the field and may be used for controlling the resistant pests.

### INTRODUCTION

MicroRNAs (miRNAs) are small, endogenous single-stranded RNAs with a length of between 16 and 27 nucleotides that regulate translation of messenger RNA (mRNA) into protein by hybridizing to the mRNA. The existence and physiological roles of miRNA in organisms of all kingdoms have been reported, where miRNAs have been shown to perform multiple and varied functions. It has been shown that miRNAs in insects such as mosquitoes play important roles in various biological processes, for example, blood digestion, ovulation, sexual differentiation, pesticide resistance, and infection with pathogens.

Resistance is a heritable change in the susceptibility of a pest population that causes the repeated failure of a pesticide to reach an appropriate level of pest control. As a result of continuous applications of the same pesticide, naturally resistant pests prevail (namely, vector population), which mate leaving offspring also resistant and becoming predominant in the population.

Monitoring resistance in the vector population is extremely valuable in determining the possible causes of pest control failures. To help delay or prevent the development of pesticide resistance in vector populations, annual monitoring of resistance in target populations allows for early detection of resistance and proper selection of pesticides.

The CDC has formulated a trial to determine whether a specific active ingredient in an insecticide can kill mosquito vectors. The technique, known as the CDC bottle bioassay, includes coating a bottle with a known amount of insecticide (that is, diagnostic dose), placing mosquitoes into the bottle, and observing the mosquitoes for two hours. Resistance is determined by the percentage of mosquitoes that die (namely, the mortality rate) at a pre-determined threshold time during those two hours. Inhibitors, such as enzyme inhibitors, can be used to determine the mechanism behind resistance by exposing mosquitoes to the inhibitors for a set period of time followed by exposure to an insecticide inside the bottles or by exposing the mosquitoes to the inhibitor and insecticide in the same bottle.

Although this assay is straightforward, it requires that the bottles are clean, dry, and coated with insecticide and/or inhibitor before each bioassay, which is time-consuming. If the bottles are contaminated before the coating, the test results will be unreliable. Another disadvantage of the CDC bottle bioassay is that it typically requires the use of a known susceptible population (a lab colony) of the same species of mosquito for an initial comparison to a field population. Furthermore, the use of inhibitors requires additional manipulation of the mosquitoes otherwise the inhibitor may not have enough time to counteract the enzymes in the mosquitoes.

Thus, there is a need for detecting the exact mechanism in pests that gives rise to pesticide resistance and a means of detecting that mechanism rapidly in the field.

### SUMMARY

In an aspect, the disclosure relates to a kit for the determination of resistance of a pest to a pesticide. An exemplary kit may comprise: a system for maceration of the pest; a primer solution comprising one or more primer pairs, wherein each primer pair binds to a specific microRNA (miRNA); and a colorimetric system for detecting the presence or absence of the specific miRNA.

In another aspect, the disclosure relates to a method of detecting resistance of pests to a pesticide. An exemplary method may include collecting a sample of pests; macerating the sample of pests; incubating a primer solution with the macerated sample of pests, the primer solution comprising one or more primer pairs, wherein each primer pair binds to a specific microRNA (miRNA); and performing a colorimetric-based detection of the miRNA.

In another aspect, the disclosure relates to a method of controlling one or more pesticide resistant pests. An exemplary method may include collecting a sample of pests; macerating the sample of pests; incubating a primer solution with the macerated sample of pests, the primer solution comprising one or more primer pairs, wherein each primer pair binds to a specific microRNA (miRNA) and the specific miRNA corresponds to a specific pesticide; performing a colorimetric-based detection of the specific miRNA; when the specific miRNA is detected, administering to the pests a pesticide different than the specific pesticide.

The disclosure provides for other aspects and embodiments that will be apparent in light of the following detailed description and accompanying figures.

### BRIEF DESCRIPTION OF THE DRAWINGS

FIG. 1 shows an exemplary process for comparing and identifying differential miRNA expression between Ae. aegypti exposed and not exposed to the insecticide CIELO™.

FIG. 2 shows mortality curves of Ae. aegypti (New Orleans strain) populations treated with CIELO™. Each curve corresponds to different concentrations of insecticide during 90 minutes of exposure.

FIG. 3 shows reading in Reads of the miRNAs found in Ae. Aegypti of the MT strain resistant to pyrethroid insecticides.

FIG. 4 shows reading in Folds of the miRNAs found in Ae. Aegypti of the MT strain resistant to pyrethroid insecticides.

FIG. 5A and FIG. 5B show the steps for identification of miRNAs in a sample using colorimetric detection.

### DETAILED DESCRIPTION

The instant disclosure relates to systems and methods for detecting resistance of pests to a pesticide. Exemplary methods and systems may be used in the field and may be used for controlling the resistant pests.

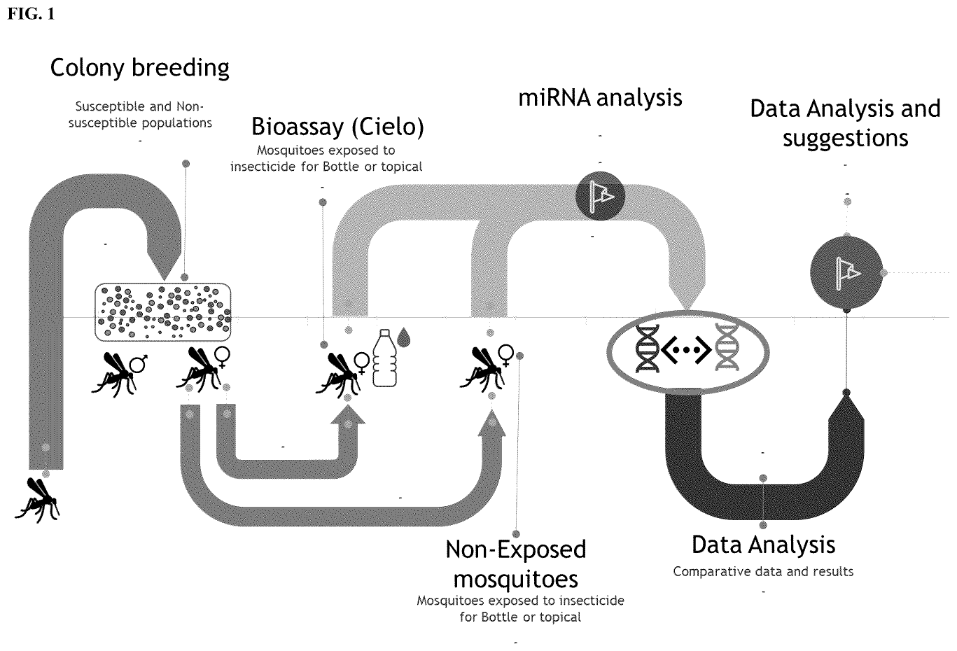

FIG. 1 shows an exemplary process for comparing and identifying differential miRNA expression between Ae. aegypti exposed and not exposed to the insecticide CIELO™. The exemplary process begins with colony breeding, which may include mosquito populations that are susceptible and not susceptible to a particular pesticide. Certain members of the colony may be selected, which may be done at random, and subjected to a bioassay test. Exemplary bioassay tests include bottle bioassays and topical bioassays. Mosquitos subjected to the bioassay, and mosquitos not subjected to the bioassay, may be subjected to miRNA analysis. Data from the miRNA analysis may be evaluated and one or more suggestions or actions items may result from the data analysis.

### 1. Definitions

Unless otherwise defined, all technical and scientific terms used here have the same meaning as commonly understood by one of ordinary skill in the art. In case of conflict, the present document, including definitions, will control. Various methods and materials are described below, although methods and materials similar or equivalent to those described here can be used in practice or testing. All publications, patents, patent applications, and/or other documents cited in this application are incorporated by reference in their entirety for all purposes to the same extent as if each individual publication, patent, patent application, and/or other document were individually indicated to be incorporated by reference for all purposes. The materials, methods, and examples disclosed are illustrative only and not intended to be limiting.

The terms “comprise(s),” “include(s),” “having,” “has,” “can,” “contain(s),” and variants thereof, as used here, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented here, whether explicitly set forth or not.

For the recitation of numeric ranges, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used here as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within three or more than three standard deviations, per the practice in the art. Alternatively, such as with respect to biological systems or processes, the term “about” can mean within an order of magnitude, preferably within five-fold, and more preferably within two-fold, of a value.

“Amino acid” as used here refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to here by either their commonly known three-letter symbols or by the one-letter symbols recommended by the IUPAC-IUB Biochemical Nomenclature Commission. Amino acids include the side chain and polypeptide backbone portions.

“Biomarker” refers to a naturally occurring biological molecule present in a subject at varying concentrations useful in predicting the risk or incidence of a condition such as insecticide resistance. The biomarker may be a small molecule, polynucleotide such mRNA or miRNA or a gene, a polypeptide or protein, lipid, or carbohydrate. For example, the biomarker can be a miRNA present in higher or lower amounts in a subject at risk for insecticide resistance. The biomarker can include nucleic acids, ribonucleic acids, or a polypeptide used as an indicator or marker for a condition in a subject. In some embodiments, the biomarker is a miRNA. A biomarker may also comprise any naturally or non-naturally occurring polymorphism (for example, single-nucleotide polymorphism [SNP]) present in a subject that is useful in predicting the risk or incidence of a condition.

“Complement” or “complementary” as used here can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The terms “control,” “reference level,” and “reference” are used interchangeably. The reference level may be a predetermined value or range, which is employed as a benchmark against which to assess the measured result. “Control group” as used refers to a group of control organisms. The predetermined level may be a cutoff value from a control group. The predetermined level may be an average from a control group. The normal levels or ranges for a target or for a miRNA activity may be defined in accordance with standard practice. A control may be an organism without insecticide resistance. A control may be an organism, or a sample therefrom, whose condition is known. The organism, or sample therefrom, may be healthy or exposed to an insecticide.

“Effective amount” refers to a dosage of a compound or composition sufficient for eliciting a desired effect, commensurate with a reasonable benefit/risk ratio. This term as used here may also refer to an amount effective at bringing about a desired in vivo effect in a subject, preferably, an insect, such as mortality after exposure to an effective amount of an insecticide.

“Identical” or “identity” as used here in the context of two or more polynucleotide or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of a single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

As used here the terms “microRNA,” “miRNA,” and “miR” are used synonymously to refer to an about 16-27 nucleotide (nt) long, non-coding RNAs derived from endogenous genes. miRNAs are processed from longer (approximately 75 nt) hairpin-like precursors termed pre-miRNAs. miRNAs assemble in ribonucleoprotein complexes termed miRNPs and recognize their targets by antisense complementarity. If the miRNAs match 100% their target, that is, the complementarity is complete, the target mRNA is cleaved, and the miRNA acts like a small interfering (siRNA). If the match is incomplete, that is, the complementarity is partial, then the translation of the target mRNA is blocked. The complementary nucleotides may be contiguous or may be interspersed with non-complementary nucleotides. The miRNA may comprise a nucleotide sequence wherein 10, 11, 12, 13, 14, 15, 16, 17, 18, 19, 20, 21, 22, 23, 24, 25, 26, or 27 nucleotides are complementary to the nucleotides in SEQ ID Nos: 1-3. The 10, 11, 12, 13, 14, 15, 16, 17, 18, 19, 20, 21, 22, 23, 24, 25, 26, or 27 complementary nucleotides may be contiguous or may be interspersed with non-complementary nucleotides.

“Mosquito” as used here refers to any species of the roughly 3,500 species of the insect that is commonly associated with and given the common name “mosquito.” Mosquitoes span 41 insect genera, including the non-limiting examples of Aedes, Culex, Anopheles (carrier of malaria), Coquillettidia, and Ochlerotatus.

“Normal gene” as used here refers to a gene that has not undergone a change, such as a loss, gain, or exchange of genetic material. The normal gene undergoes normal gene transmission and gene expression. For example, a normal gene may be a wild-type gene.

“Nucleic acid” or “oligonucleotide” or “polynucleotide” as used here means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and their complements. A single strand provides a probe that may hybridize to a target sequence under stringent hybridization conditions. Thus, a polynucleotide also encompasses a probe that hybridizes under stringent hybridization conditions. Polynucleotides may be single stranded or double stranded or may contain portions of both double stranded and single stranded sequence. The polynucleotide can be nucleic acid, natural or synthetic, DNA, genomic DNA, cDNA, RNA, miRNA, or a hybrid, where the polynucleotide can contain combinations of deoxyribo- and ribo-nucleotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

A “peptide” or “polypeptide” is a sequence of two or more amino acids linked by peptide bonds. The polypeptide can be natural, synthetic, or a modification or combination of natural and synthetic. Peptides and polypeptides include proteins such as binding proteins, receptors, and transport proteins. The terms “polypeptide,” “protein,” and “peptide” are used interchangeably. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. A domain may be comprised of a series of the same type of motif.

“Pest” refers to small organisms that are capable of destroying crops, food, causing diseases, and attacking other organisms. The pest may be of the order Diptera such as mosquitoes, Nematocera (e.g., crane flies, midges, gnats), Brachycera (e.g., horse flies, robber flies, bee flies), and Cyclorrhapha (e.g., flies that breed in living or dead vegetable or animal material).

As used here, the term “pesticide” includes insecticides. A pesticide may also be referred to as a “toxin.” Pesticides may be any pesticide that is capable of acting as such and to which resistance has been identified amongst the, or some of the, pest(s) against which it is otherwise active.

As used here, “pesticide resistance” refers to an overall reduction in the ability of a pesticide to kill a pest. Meaning that, when a pesticide is used as directed, the pesticide no longer works or only partially works. Pesticide resistance can be pesticide specific, or it can develop to certain class(es) of pesticide(s). Over time, the repeated use of pesticide(s) can lead to pesticide resistance in pest populations. Proteins are produced and/or activated to help the process of detoxification of pesticides where miRNA dictates the overall efficiency of the detoxifying activity by regulating the proteins. There are three primary forms of resistance that involve miRNA: modifications to protein structure (that is, knockdown resistance; “KDR”); proteins specialized in detoxification that enable a pest to detoxify or destroy the toxin faster than susceptible pests or prevent the toxin from reaching target sites (that is, metabolic resistance); and, proteins involved in organ and tissue development (that is, behavior/chitinic resistance).

“Sample” or “test sample” as used here can mean any sample in which the presence and/or level of a target such as a biomarker is to be detected or determined or any sample comprising a vector. The sample may be a biological sample. Samples may include liquids, solutions, emulsions, or suspensions. Samples can be obtained by any means known in the art. The sample can be used directly as obtained from an organism or can be pre-treated, such as, by filtration, distillation, extraction, concentration, centrifugation, inactivation of interfering components, and addition of reagents, to modify the character of the sample in some manner as discussed here or otherwise as is known in the art.

“Subject” and “organism” as used here interchangeably refer to any insect or pest, including, but not limited to, any insect or pest which is known to offer at least some resistance to a pesticide or insecticide, and which is considered necessary to disable and/or kill. Examples include those that attack or damage or otherwise reduce the commercial or other value of a substrate, such as, crops, particularly arable crops, such as, food and material crops, such as, cotton. Other pests include those that are a nuisance to or an adversary of other living organisms, including mammals, such as humans. For example, the insect may be a mosquito. The subject may be at any stage of development, for example, egg, larva, pupa, or imago (adult) stages. In some embodiments, the subject has a specific genetic marker.

“Substantially identical” can mean that a first and second amino acid or polynucleotide sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Treatment” or “treating” as used here refers to applying a toxin or pesticide to a subject as a means of inducing damage or death due to exposure to the toxin or pesticide. A treatment may be either performed in an acute or chronic way.

“Variant” with respect to a polynucleotide means (i) a portion or fragment of a referenced nucleotide sequence; (ii) the complement of a referenced nucleotide sequence or portion; (iii) a nucleic acid that is substantially identical to a referenced nucleic acid or its complement; or (iv) a nucleic acid that hybridizes under stringent conditions to the referenced nucleic acid, its complement, or a sequence substantially identical.

Unless otherwise defined here, scientific and technical terms used in connection with the present disclosure shall have the meanings that are commonly understood by those of ordinary skill in the art. For example, any nomenclatures used in connection with, and techniques of, cell and tissue culture, molecular biology, microbiology, genetics, and protein and nucleic acid chemistry and hybridization described here are those that are well known and commonly used in the art. The meaning and scope of the terms should be clear; in the event however of any latent ambiguity, definitions provided here take precedent over any dictionary or extrinsic definition. Further, unless otherwise required by context, singular terms shall include pluralities and plural terms shall include the singular.

### 2. Exemplary Kits

Described here are kits that may be used to detect resistance of a pest to a pesticide.

The kit may comprise a system for maceration of the pest. In some embodiments, the system for maceration may comprise a vial, a sterile pestle, and macerating solutions/buffers. The maceration system may be used as follows: pests are placed in the vial, the maceration buffer is added to the vial comprising the pests, and the pestle is repeatedly pushed into/against the vial and rotated in the vial to create a homogenous mixture.

The kit may further comprise a primer solution comprising one or more primer pairs, wherein each primer pair binds to a specific miRNA. The primer solution may be added to the vial comprising the macerated pests or the pests may be added to a vial comprising the primer solution and then macerated in the vial.

The primer solution may comprise any component necessary for amplification of nucleic acids from the miRNA required for detection of the miRNA. For example, the primer solution may comprise, dNTPs, reverse transcriptase, polymerase enzymes, and magnesium chloride.

The primer solution may aid in reverse transcription of the miRNA into complementary DNA (cDNA) and amplification of the cDNA for colorimetric detection of the miRNA where the primer solution produces fluorescence when the specific miRNA is present in the sample. The composition of the primer solution may be adjusted in accordance with various factors including the type of pest, the species of pest, the type of pesticide, and the primers.

The miRNA to be targeted by the primers may be identified by exposing a pest to a pesticide in a controlled setting, processing and extracting RNA from the pest by any means known in the art, performing sequencing analyses on the extracted RNA such as next-generation sequencing, and comparing the sequencing results between pesticide resistant and non-resistant pests to identify differences in miRNA expression. The miRNA may be known to contribute to pesticide resistance or the role of the miRNA in pesticide resistance may be unknown. The primers may be designed to target miRNA that is expressed in resistant pests at significantly greater levels than in non-resistant pests.

The role of miRNA typically is to downregulate expression of mRNA that the miRNA binds to. Therefore, the downregulation mRNA site may be detected. If this downregulation mRNA site changes, then the change can be detected by comparing similar runs. The primers may bind to a portion of the miRNA. The primers may be from about 16 to about 40 nucleotides in length. The primers may comprise a forward primer and a reverse primer pair that targets a specific miRNA. The primers in the primer pair may have a similar melting temperature (Tₘ), such as within 10° C. of each other, or within 5° C. of each other.

Examples of suitable pesticides may include sodium channel modulators (such as pyrethroids, pyrethrins, and DDT), nicotinic acetylcholine receptor competitive modulators (such as neoniconitoids, imidacloprids, sulfoximines, nicotine, butenolides, mesoionics, nicotinoids, and pyridylidenes), acetylcholinesterase inhibitors (such as organophosphates and carbamates), GABA-gated chloride channel blockers (such as organochlorines and phenylpyrazoles), organosulfurs, formamidines, dinitrophenols, organotins, nicotinic acetylcholine receptor allosteric modulators—Site I (such as spinosyns), modulators of mitochondrial electron transport (such as pyrazoles, pyridazinones), modulators of chitin synthesis (such as quinazolines and benzoylureas), botanicals (such as pyrethrum, nicotine, rotenone, limonene, and neem), synergists/activators that inhibit cytochrome P-450 dependent polysubstrate monooxygenases, antibiotics, fumigants, inorganics (such as arsenicals, sulfur, and boric acid), biorational (such as hormones, enzymes, pheromones natural agents such as growth regulators, and microbials), glutamate-gated chloride channel allosteric modulators (such as avermectins), and combinations thereof. In an embodiment, the pesticide may be a pyrethroid, a neonicotinoid, an organophosphate, or combination thereof. In a particular embodiment, the pesticide may be a pyrethroid, a neonicotinoid, or combination thereof.

It will be appreciated that new pesticides and new classes of pesticides are discovered from time to time, and that resistance to pesticides can develop over time. Although the mechanism of resistance may differ, resistance may develop to any pesticide (Buhler, Wayne. “Understanding Resistance.” Pesticide Environmental Stewardship, pesticidestewardship.org; “Slowing and Combating Pest Resistance to Pesticides.” Environmental Protection Agency, epa.gov). It is intended that the principles of this disclosure, and the concepts here, can be applied to a wide range of pesticides, both known and those yet to be discovered, and when resistance is identified.

The kit may also include instructions for and a means for detecting the presence or the absence of one or more miRNAs. The means for detecting the presence or the absence of one or more miRNAs may comprise a colorimetric system. The colorimetric system may comprise any rapid colorimetric assay known in the art. For example, the colorimetric assay may be Nicking Enzyme Signal Amplification (NESA), Nucleic Acid Sequence Based Amplification (NASBA), loop-mediated isothermal amplification (LAMP), or reverse-transcription loop-mediated isothermal amplification (RT-LAMP).

The colorimetric system may comprise a paper test strip for enzymatic detection of resistance. For example, paper test strips may be impregnated with a buffer solution and dried, then the test strips may be used to detect specific enzymes such as monooxygenases involved in resistance by a color change of the test strip following exposure to the enzymes. The paper test strip may be dipped into a vial comprising the primer solution and macerated pests and incubated for seconds until a change in color is detected. The colorimetric system may produce a visible color change in at most 15 minutes. For example, the visible color change may be observed within about five minutes to about 15 minutes.

Instructions included in kits may be affixed to packaging material or may be included as a package insert. While the instructions are typically written on printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used here, the term “instructions” may include the address of an internet site that provides the instructions.

### 3. Methods

a. Methods of Identifying miRNAs in a Sample

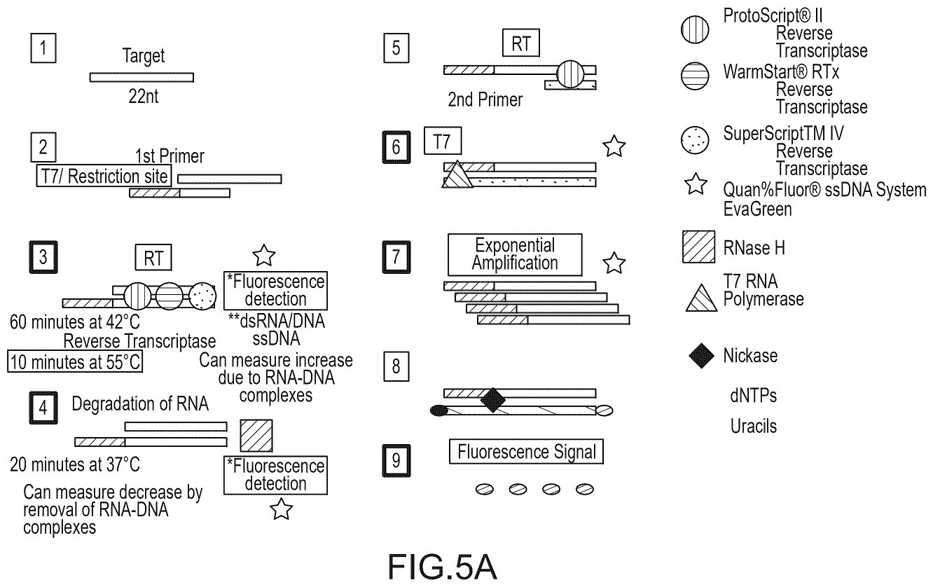

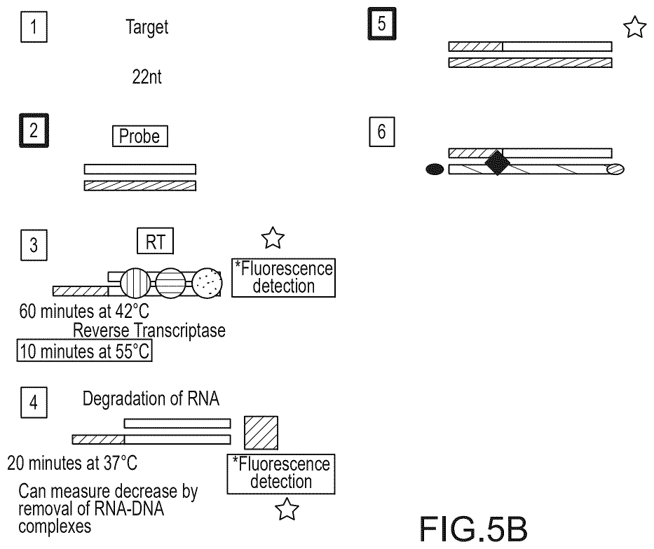

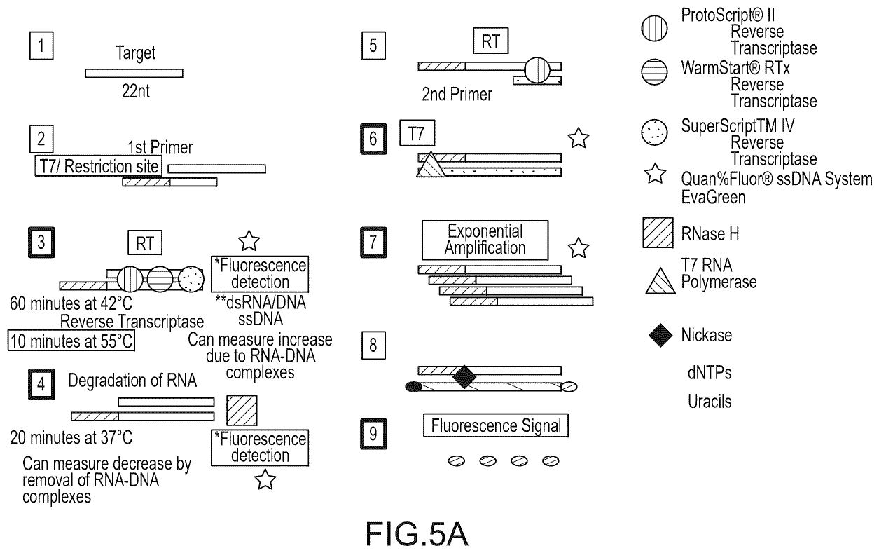

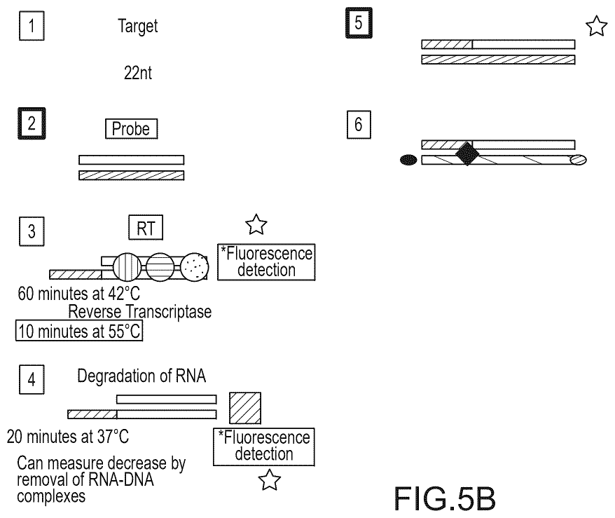

FIG. 5A and FIG. 5B show various operations for identification of miRNAs in a sample using primers and/or probes. The process shown in FIG. 5B may be used for knock down resistance (KDR) detection using isothermal processing. A vial comprising a primer solution in which a pest is macerated is used to generate cDNA and produce a fluorescence signal for the detection of miRNA. Each step having a bolded outline is an opportunity for colorimetric detection. There are five points for potential colorimetric detection of miRNA in FIG. 5A. Detection of miRNA occurs when fluorescence is present at operation 3. However, to reduce error, four additional points of colorimetric detection of the miRNA are included. If all of the colorimetric steps produce a fluorescent signal, then operation 9 of FIG. 5A will show the desired fluorescence indicative of the presence of specific miRNAs in the sample.

Operation 1 includes extracting the baseline and selecting specific MiRNAs. Operation 2 includes locating the selected sites and annealing to a restriction site with the assistance of the first primer. Operation 3 includes incubation of reverse transcriptase enzymes for colorimetric detection. Operation 4 involves carrying out RNA degradation, whereby negative colorimetry may be used. Results in Operation 4 may be measured for a reduction in fluorescence using an RNA enzyme. Operation 5 involves using reverse transcriptase on a second primer in a specific resulting RNA segment. Operation 5 may assist with Operation 6. Operation 6 includes preparing copies of double stranded RNA (dsRNA). Operation 6 may include exponential amplification facilitated by RNA polymerase. Operation 7 includes exponential or massive amplification of enzymes through temperature cycles to filter and finalize fluorescence detection. Operation 8 includes validation with the usage of Nickase that may dislodge an RNA to promote fluorescence. Operation 9 includes a signal strength measurement.

b. Methods of Detecting Resistance of Pests to a Pesticide

Described here are methods of detecting resistance of pests to a pesticide. The methods may include collecting a sample of pests in a vial; macerating the sample of pests as described here; incubating a primer solution comprising one or more primer pairs as described here with the sample of pests, wherein each primer pair binds to a specific miRNA as described here; and, performing a colorimetric-based detection of the miRNA as described here.

In some embodiments, the miRNA is expressed at higher levels in a pesticide resistant pest than a pest that is susceptible to the pesticide. In some embodiments, the miRNA is expressed at lower levels in a pesticide resistant pest than a pest that is susceptible to the pesticide. In some embodiments, the sample of pests may be resistant to the pesticide if the colorimetric-based detection results in a color change. In some embodiments, the method may detect metabolic resistance or knockdown resistance in the pests.

c. Methods of Controlling Pesticide Resistant Pests

Described here are methods of controlling one or more pesticide resistant pests. The methods may include collecting a sample of pests in a vial; macerating the sample of pests as described here; incubating a primer solution comprising one or more primer pairs as described here with the sample of pests, wherein each primer pair binds to a specific miRNA as described here and the miRNA corresponds to a specific pesticide; performing a colorimetric-based detection of the miRNA as described here; and if the miRNA is detected, then a pesticide different than the specific pesticide may be administered to the pests.

In some embodiments, administration of the pesticidal composition provides droplets having an average diameter of less than 30 μm. In some embodiments, the pesticide may be applied as an aerosol or fog, wherein the aerosol or fog contacts the population of pests. In some embodiments, the methods described here can comprise any known route, apparatus, and/or mechanism for the delivery or application of the pesticide. In some embodiments, the method comprises a sprayer. Traditional pesticide sprayers in the pest control markets are typically operated manually or electrically or are gas-controlled and use maximum pressures ranging from 15 to 500 psi generating flow rates from 5 gpm to 40 gpm. In some embodiments, the methods disclosed here comprise the use of pesticides in combination with any low volume environmental pest control device(s) such as, for example, ultra-low volume (ULV) machines. Such combinations are useful in methods for flying pest control (e.g., flies, gnats, flying ants, and sand fleas) wherein contacting the pest with a low volume of the pesticide is possible and/or desirable. ULV machines use low volume of material, for example, at rates of about one gallon per hour (or ounces per minute), and typically utilize artificial wind velocities such as from, for example, an air source (e.g., pump or compressor) to break down and distribute the composition/formulation into a cold fog (e.g., having average droplet particle sizes of about 1-30 μm). Any standard ground ULV equipment used for adult mosquito control such as, for example, a system including a (CETI) aerosol generator can be used in the methods described here. A general ULV system includes a tank for the pesticidal composition, a transport system (e.g., a pump or pressurized tank), a flow control device, and a nozzle that atomizes the composition. Typically, ULV machines do not compress droplets. Rather, they often use a venturi siphoning system and can induce an artificial energizing of the droplets by adding an electrical current to the liquid (e.g., through the use an electrode located at the application tip).

### 4. EXAMPLES

The foregoing may be better understood by reference to the following examples, which are presented for purposes of illustration and are not intended to limit the scope of the disclosure. The present disclosure has multiple aspects and embodiments, illustrated by the appended non-limiting examples.

### Example 1

### Insecticide Resistance Determination

Based on the CDC bottle bioassay protocol (CONUS Manual for Evaluating Insecticide Resistance in Mosquitoes Using the CDC Bottle Bioassay Kit, CDC 2020), six different doses of insecticide were evaluated. For this evaluation, 20 females of Ae. aegypti (3-5 days) per bottle of 250 mL were used and replicated three times for each of the doses. Doses ranging from a minimum mortality of 0% to a maximum mortality of 100% were selected. The bioassays were performed on the New Orleans strain (NO), which was kept under laboratory conditions with an oscillating temperature of 22° C.±2 and relative humidity of 60%. The CDC bioassay (exclusive dose 1 μg/bottle) was repeated with the Martínez de la Torre, Veracruz, Mexico strain (MT) previously reported as resistant to pyrethroids type I and II. MT were processed to obtain total RNA with the Phenol-Chloroform extraction technique, and the nucleic acids were stored dry at a temperature of −20° C. until their shipment to the company BGI Genomics, which sequenced the small fraction of next-generation RNAs.

The results of the bottle bioassays are shown in TABLE 1. the mortalities of female mosquitoes of the NO strain were observed before the insecticide CIELO™ (available from Clarke Mosquito Control Products, St. Charles, IL) (which includes Imidacloprid+Pyrethroid) during one hour of exposure to six different doses.

TABLE 1
Dosage and mortality of Ae. aegypti before the insecticide
CIELO ™ during one hour of exposure.

| Strain | Insecticide | Concentration | Exposed | % Mortality |
|---|---|---|---|---|
| NO | CIELO ™ | 1 | 60 | 57.33 |
| NO | CIELO ™ | 2.5 | 60 | 68 |
| NO | CIELO ™ | 5 | 60 | 65 |
| NO | CIELO ™ | 10 | 60 | 74.66 |
| NO | CIELO ™ | 15 | 60 | 100 |
| NO | CIELO ™ | 25 | 60 | 100 |

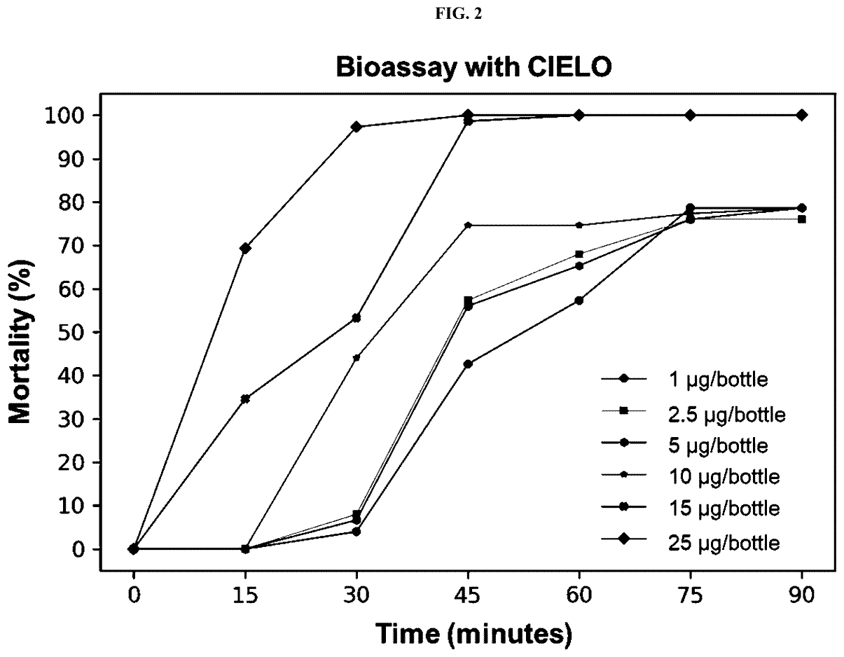

FIG. 2 shows the doses and mortalities up to 90 minutes of exposure to CIELO™ and an overview of the doses of CIELO™. The doses corresponding to 15 μg/bottle and 25 μg/bottle reached 100% mortality at 45 min, on the other hand, the remaining doses did not reach 100% of mortality even after 90 min of exposure.

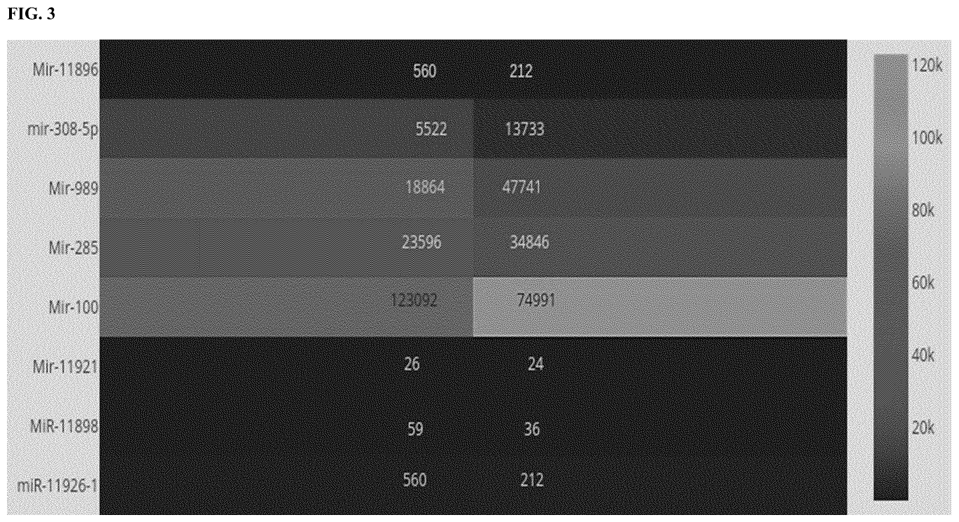

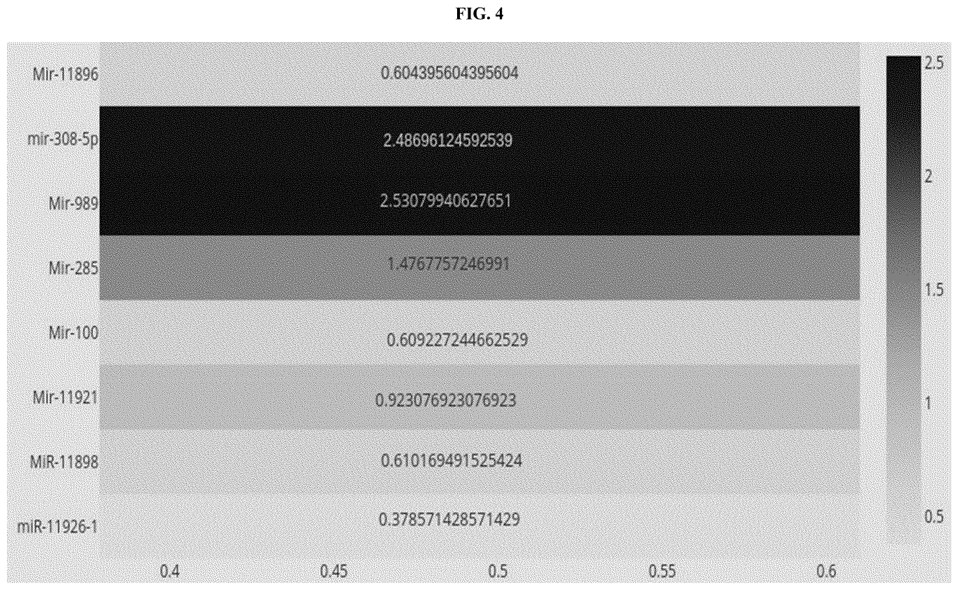

The results of the sequencing can be seen in FIG. 3 and FIG. 4, where the heatmaps show the Reads (FIG. 3) of the microRNAs and the Folds (FIG. 4). As the names reflect, the Reads correspond to the number of readings of the sequences that were carried out when analyzing the MirDeep data, and the Folds are interpreted as the quotient between readings.

The Reads are readings made by the MirDeeptool, each reading is equivalent to a mating to the genome; therefore, the more readings the greater the interaction with the genome.

FIG. 3 shows reading in Reads of the miRNAs found in Ae. Aegypti of the MT strain resistant to pyrethroid insecticides, the two columns correspond to mosquitoes exposed to CIELO™ (first column) and unexposed mosquitoes (second column). The Reads in this section indicate the number of times the miRNAs mated with the mosquito genome, recalling that there were two different situations causing a different number of mating or readings (Reads) in the same mosquito strain.

On the other hand, the Fold's are a flexible unit of measurement, the user can manipulate the magnitudes depending on the desired degree of contrast. Here, the Folds are adjusted to the value of 1, that is, how many times it meets the value of one of itself in other microRNAs.

FIG. 4 shows reading in Folds of the miRNAs found in Ae. Aegypti of the MT strain resistant to pyrethroid insecticides, two molecules are seen that contrast between strains, these molecules correspond to mir-308-5p and mir-989. The contrast is measured by the Folds,—the molecules of Mir-308-5p are found 2.48 times more in unexposed mosquitoes than in exposed mosquitoes and in Mir-989 molecules are found 2.53 times more in unexposed mosquitoes than in exposed mosquitoes. The greater the contrast, the higher is the level of interest.

### Example 2

Identification of miRNA Involved in Resistance

A bioinformatic analysis was performed from Next Generation Sequencing (NGS) results, using the miRDeep2 bioinformatic tool (Friedländer, et. al., 2012), which identifies known and new miRNAs in the sequencing data. To verify that the results were exclusive miRNA of Ae. aegypti, all of the runs were compared against the miRBase database (mirbase.org; Kozomara, Birgaoanu & Griffiths-Jones, 2019).

With the above, it was possible to determine a candidate miRNA (aae-miR-11898, Aedes aegypti specific) to be used in the Nicking enzyme signal amplification (NESA) and Nucleic Acid Sequence Based Amplification (NASBA) protocol, as it presents high levels of expression in the resistant strain (349488 reads).

The sequence for aae-miR-1 is: AUAAGUGGGGCAAAGACACCAA (SEQ ID NO: 1). The primers used for miRNA detection are shown in TABLE 2.

TABLE 2
Primers for miRNA.

| | | SEQ | length | GC | Tm |
|---|---|---|---|---|---|
| Primer | Sequence | ID NO: | (bp) | (%) | (° C.) |
| Forward | GGACGGTAGCAAGCAAAGAGTGTGTT | 2 | 35 | 48.57 | 71.93 |
| | GGTGTCTTT | | | | |
| Reverse | GGGATTCTGGAAGATGATGATGACAT | 3 | 35 | 48.57 | 69.93 |
| | AAGTGGGGC | | | | |

To synthesize the primers, an oligo synthesizer was used (biolytic.com/t-dna-rna-oligo-synthesizer-768xlc.aspx), and the primers were acquired through a commercial distributor and primer designer company. The primers are comprised of a cocktail composition based on a “master mix” that contains the necessary substrates to carry out the reaction, such as, polymerase enzyme and magnesium chloride. This composition was the test base for the aae-miR-1 sequence described above. The composition is based on the primer design described above and then a sample collected in the field will be added to the primer+cocktail composition.

The miRNA sequence and design are unique and exclusive to the technician performing the mirnomic process under laboratory-controlled conditions; therefore, the primers that are designed from the original sequence (miRNA), will also be de novo. The entire design has been made in-silico (through a computer software) from the sequences found after Aedes aegypti has been exposed to an insecticide, in this case CIELO™.

The foregoing description of the specific aspects will so fully reveal the general nature of the technology that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented here. It is to be understood that the phraseology or terminology is for the purpose of description and not of limitation such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

For reasons of completeness, various aspects of the technology are set out in the following numbered clauses:

Clause 1. A kit for determining resistance of a pest to a pesticide, the kit comprising:
a system for maceration of the pest;
a primer solution comprising one or more primer pairs, wherein each primer pair binds to a specific microRNA (miRNA); and,
a colorimetric system for detecting the presence or absence of the specific miRNA.
   Clause 2. The kit according to Clause 1, wherein the system for maceration comprises at least one of a vial, a sterile pestle, and macerating solution.
   Clause 3. The kit according to Clause 1 or Clause 2, wherein the colorimetric system is configured to detect expression of miRNA at higher levels in a pesticide resistant pest than a pesticide susceptible pest.
   Clause 4. The kit according to any one of Clauses 1-3, wherein the colorimetric system comprises Nicking Enzyme Signal Amplification (NESA) or Nucleic Acid Sequence Based Amplification (NASBA).
   Clause 5. A method of detecting resistance of pests to a pesticide, the method comprising: collecting a sample of pests;
macerating the sample of pests;
incubating a primer solution with the macerated sample of pests, the primer solution comprising one or more primer pairs, wherein each primer pair binds to a specific microRNA (miRNA); and
performing a colorimetric-based detection of the miRNA.
   Clause 6. The method of Clause 5, wherein performing the colorimetric-based detection comprises detecting expression of the miRNA at higher levels in a pesticide resistant pest than a pesticide susceptible pest.
   Clause 7. The method of Clause 5 or Clause 6, further comprising determining that the sample of pests are resistant to the pesticide when the colorimetric-based detection results in a color change.
   Clause 8. The method of any one of Clauses 5-7, wherein the resistance is metabolic resistance or knockdown resistance.
   Clause 9. The method of any one of Clauses 5-8, further comprising detecting a visible color change during the colorimetric-based detection within at most 15 minutes.
   Clause 10. A method of controlling one or more pesticide resistant pests, the method comprising:
collecting a sample of pests;
macerating the sample of pests;
incubating a primer solution with the macerated sample of pests, the primer solution comprising one or more primer pairs, wherein each primer pair binds to a specific microRNA (miRNA) and the specific miRNA corresponds to a specific pesticide;
performing a colorimetric-based detection of the specific miRNA;
when the specific miRNA is detected, administering to the pests a pesticide different than the specific pesticide.
   Clause 11. The method of Clause 10, further comprising detecting the specific miRNA when the colorimetric-based detection results in a color change.
   Clause 12. The method of Clause 10 or Clause 11, wherein the resistance is metabolic resistance or knockdown resistance.
   Clause 13. The method of any one of Clauses 10-12, further comprising detecting a visible color change during the colorimetric-based detection in at most 15 minutes.

SEQUENCES

SEQ ID NO: 1
aae-miR-1 from Aedes aegypti
AUAAGUGGGGCAAAGACACCAA
SEQ ID NO: 2
Forward Primer for aae-miR-1
GGACGGTAGCAAGCAAAGAGTGTGTTGGTGTCTTT
SEQ ID NO: 3
Reverse Primer for aae-miR-1
GGGATTCTGGAAGATGATGATGACATAAGTGGGGC

1. A kit for determining resistance of a pest to a pesticide, the kit comprising:
a system for maceration of the pest;
a primer solution comprising one or more primer pairs, wherein each primer pair binds to a specific microRNA (miRNA); and,
a colorimetric system for detecting the presence or absence of the specific miRNA.

2. The kit according to claim 1, wherein the system for maceration comprises at least one of a vial, a sterile pestle, and macerating solution.

3. The kit according to claim 2, wherein the colorimetric system is configured to detect expression of miRNA at higher levels in a pesticide resistant pest than a pesticide susceptible pest.

4. The kit according to claim 3, wherein the colorimetric system comprises Nicking Enzyme Signal Amplification (NESA) or Nucleic Acid Sequence Based Amplification (NASBA).

5. A method of detecting resistance of pests to a pesticide, the method comprising:
collecting a sample of pests;
macerating the sample of pests;
incubating a primer solution with the macerated sample of pests, the primer solution comprising one or more primer pairs, wherein each primer pair binds to a specific microRNA (miRNA); and
performing a colorimetric-based detection of the miRNA.

6. The method of claim 5, wherein performing the colorimetric-based detection comprises detecting expression of the miRNA at higher levels in a pesticide resistant pest than a pesticide susceptible pest.

7. The method according to claim 6, further comprising determining that the sample of pests are resistant to the pesticide when the colorimetric-based detection results in a color change.

8. The method according to claim 7, wherein the resistance is metabolic resistance or knockdown resistance.

9. The method according to claim 8, further comprising detecting a visible color change during the colorimetric-based detection within at most 15 minutes.

10. A method of controlling one or more pesticide resistant pests, the method comprising:
collecting a sample of pests;
macerating the sample of pests;
incubating a primer solution with the macerated sample of pests, the primer solution comprising one or more primer pairs, wherein each primer pair binds to a specific microRNA (miRNA) and the specific miRNA corresponds to a specific pesticide;
performing a colorimetric-based detection of the specific miRNA;
when the specific miRNA is detected, administering to the pests a pesticide different than the specific pesticide.

11. The method according to claim 10, further comprising detecting the specific miRNA when the colorimetric-based detection results in a color change.

12. The method according to claim 11, wherein the resistance is metabolic resistance or knockdown resistance.

13. The method according to claim 12, further comprising detecting a visible color change during the colorimetric-based detection in at most 15 minutes.
